(19) United States
(12) Patent Application Publication
(10) Pub. No.: US 2022/0310232 A1
(43) Pub. Date: Sep. 29, 2022

(54) SYSTEMS AND METHODS FOR GENERATING AN ARTHRITIC DISORDER NOURISHMENT PROGRAM

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/216,190

(22) Filed: Mar. 29, 2021

(51) Int. Cl. G16H 20/60; G06N 20/00; G16H 10/20

(52) U.S. Cl. CPC G16H 20/60; G06N 20/00; G16H 10/20

(57) ABSTRACT

A system for generating an arthritic disorder nourishment program includes computing device configured to obtain an arthritic element, produce an arthritic batch as a function of the arthritic element, wherein producing the arthritic batch further comprises identifying an arthritic group as a function of a medical database, and determining the batch as a function of the arthritic group and the arthritic element using an arthritic machine-learning model, determine an edible as a function of the arthritic batch, and generate a nourishment program as a function of the edible.

### FIELD OF THE INVENTION

The present invention generally relates to the field of artificial intelligence. In particular, the present invention is directed to systems and methods for generating an arthritic disorder nourishment program.

### BACKGROUND

Current edible suggestion systems do not account for the presence of one or more arthritis related circumstances. This leads to inefficiency of an edible suggestion system and a poor nutrition plan for the individual. This is further complicated by a lack of uniformity of nutritional plans, which results in dissatisfaction of individuals.

### SUMMARY OF THE DISCLOSURE

In an aspect a system for generating an arthritic disorder nourishment program includes a computing device configured to obtain an arthritic element, produce an arthritic batch as a function of the arthritic element, wherein producing the arthritic batch further comprises identifying an arthritic group as a function of a medical database, and determining the batch as a function of the arthritic group and the arthritic element using an arthritic machine-learning model, determine an edible as a function of the arthritic batch, and generate a nourishment program as a function of the edible.

In another aspect a method for generating an arthritic disorder nourishment program includes obtaining, by a computing device, an arthritic element, producing, by the computing device, an arthritic batch as a function of the arthritic element, wherein producing the arthritic batch further comprises identifying an arthritic group as a function of a medical database, and determining the batch as a function of the arthritic group and the arthritic element using an arthritic machine-learning model, determining, by the computing device, an edible as a function of the arthritic batch, and generating, by the computing device, a nourishment program as a function of the edible.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating an arthritic disorder nourishment program;

FIG. 2 is a block diagram of an exemplary embodiment of a range of motion according to an embodiment of the invention;

FIG. 3 is a block diagram of an exemplary embodiment of an edible directory according to an embodiment of the invention;

FIG. 4 is a block diagram of an exemplary embodiment of an arthritic timeline according to an embodiment of the invention;

FIG. 5 is a block diagram of an exemplary embodiment of a machine-learning module;

FIG. 6 is a process flow diagram illustrating an exemplary embodiment of a method of generating an arthritic disorder nourishment program; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating an arthritis disorder nourishment program. In an embodiment, this disclosure obtains an arthritic element from an individual. Aspects of the present disclosure can be used to produce an arthritic batch. This is so, at least in part, because the disclosure incorporates a machine-learning model. Aspects of the present disclosure can also be used to determine an edible as a function of the arthritic batch. Aspects of the present disclosure allow for generating a nourishment program. Exemplary embodiments illustrating aspects of the present disclosure are described below in the context of several specific examples.

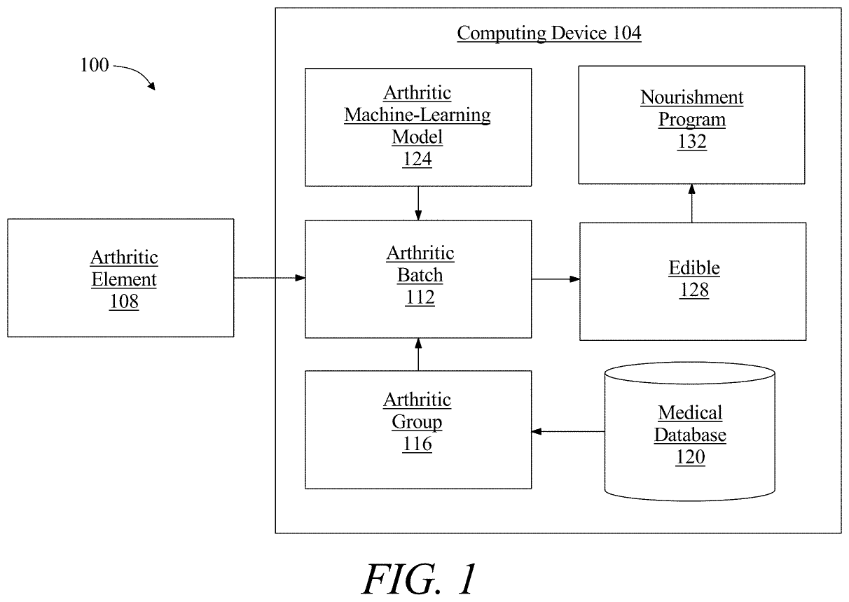

Referring now to FIG. 1, an exemplary embodiment of a system 100 for generating an arthritic disorder nourishment program is illustrated. System includes a computing device 104. computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. computing device 104 may include a single computing device operating independently, or may include two or more computing device operating in concert, in parallel, sequentially or the like; two or more computing devices may be included together in a single computing device or in two or more computing devices. computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 obtains an arthritic element 108. As used in this disclosure an “arthritic element” is an element of data associated with an individual's biological system that denotes an arthritic state, wherein an arthritic state is a measure of the relative level of physical well-being of an individual's joints and/or connective tissues. In an embodiment arthritic element may include a genetic element. As used in this disclosure a “genetic element” is an element of data associated with the composition of DNA unique to each individual. For example, and without limitation, genetic element may include an element of data denoting the individual has a predisposition for arthritis as a function of one or more genes such as, but not limited to, HLA-DRB1, HLA-B, HLA-DPB1, IRF5, RBPJ, RUNX1, and the like thereof. As a further non-limiting example, genetic element may include an element of data denoting the mutation of one or more genes associated with arthritis such as, but not limited to, STAT4, TRAF1, C5, and/or PTPN22. Arthritic element 108 may include a biological sample. As used in this disclosure a “biological sample” is one or more biological specimens collected from an individual. Biological sample may include, without limitation, exhalate, blood, sputum, urine, saliva, feces, semen, and other bodily fluids, as well as tissue. Arthritic element 108 may include a biological sampling device. Arthritic element 108 may include one or more biomarkers. As used in this disclosure a “biomarker” is a molecule and/or chemical that identifies the status of an individual's health system. As a non-limiting example, biomarkers may include, RF factor, anticyclic citrullinated peptide, C-reactive protein, erythrocyte sedimentation rate, antinuclear antibody, and the like thereof. As a further non-limiting example, arthritic element 108 may include datum from one or more devices that collect, store, and/or calculate one or more lights, voltages, currents, sounds, chemicals, pressures, and the like thereof that are associated with the individual's health status. For example, and without limitation a device may include a/an magnetic resonance imaging device, magnetic resonance spectroscopy device, x-ray spectroscopy device, computerized tomography device, ultrasound device, electroretinogram device, electrocardiogram device, ABER sensor, mass spectrometer, and the like thereof.

Still referring to FIG. 1, computing device 104 may obtain arthritic element 108 by receiving an arthritic questionnaire from a user. As used in this disclosure “arthritic questionnaire” is a data structure or display element that is provided to the user and which prompts the user to enter information germane to potential arthritic complaints; arthritic questionnaire may be provided as a function of one or more communication methods. For example and without limitation, a communication method may include a webpage and/or online questionnaire; online questionnaire may be provided using one or more form elements such as text entry boxes, buttons, checkboxes, drop-down lists, sliders, dials, and/or other display elements usable to select or enter one or more numerical values, options, identifications of body parts, sensations, range of motion information, or the like. As a further non-limiting example, communication method may include one or more applications on a cellphone, tablet, computer, game console, and the like thereof. As a further non-limiting example, communication method may include one or more methods that exist outside of a digital form of communication, such as written communication and/or verbal communication. As a non-limiting example arthritic questionnaire may include a questionnaire and/or survey that identifies a feeling of pain, joint pain, joint swelling, joint locking, reduced range of motion, headache, fever, lethargy, loss of appetite, stiffness, tenderness, malaise, redness, difficulty walking, muscle weakness, and the like thereof. Arthritic questionnaire may include one or more questionnaires and/or surveys from an informed advisor as a function of a medical assessment, wherein a “medical assessment” is an evaluation and/or estimation of the individual's health system. As used in this disclosure “informed advisor” is an individual that is skilled in the health and wellness field. As a non-limiting example an informed advisor may include a medical professional who may assist and/or participate in the medical treatment of an individual's health system including, but not limited to, family physicians, primary care physicians, orthopedists, rheumatologists, neurologists, dermatologists, occupational therapists, psychologists, psychiatrists, infectious disease physicians, geneticists, physical therapists, and the like thereof. As a non-limiting example input may include an informed advisor that enters a medical assessment comprising a physical exam, neurologic exam, blood test, imaging test, and the like thereof. In an embodiment, and without limitation, arthritic questionnaire may include one or more questionnaires and/or surveys from a family member. For example, and without limitation, a brother, sister, mother, father, cousin, aunt, uncle, grandparent, child, friend, and the like thereof may enter to computing device 104 that an individual is has a decreased range of motion and/or is experiencing joint stiffness.

Still referring to FIG. 1, computing device 104 produces an arthritic batch 112 as a function of the arthritic element 108. As used in this disclosure an “arthritic batch” is a profile and/or estimation of an individual's joints and/or connective tissues. For example, and without limitation, arthritic batch 112 may denote that an individual's shoulder and knees are swollen and/or have a limited range of motion. As a further non-limiting example, arthritic batch 112 may denote that an individual's cartilage is deteriorating and/or eliminated from a metacarpal joint. Computing device 104 produces arthritic batch 112 as a function of identifying an arthritic group 116. As used in this disclosure an “arthritic group” is a group of cells, tissues, and/or structures that are joined together to form one or more joints and/or joint functions. For example, and without limitation, arthritic group 116 may include one or more groups such as a hip, knee, ankle, foot, metatarsophalangeal joint, interphalangeal joint, shoulder, elbow, wrist, metacarpophalangeal joint, and the like thereof. As a further non-limiting example, arthritic group 116 may include one or more groups such as, cartilage, synovial membranes, ligaments, tendons, bursas, synovial fluids, menisci, and the like thereof. As a further non-limiting example, arthritic group 116 may include one or more groups such as ball and socket joints, hinge joints, condyloid joints, pivot joints, gliding joints, saddle joints, and the like thereof. As a further non-limiting example, arthritic group 116 may include one or more groups such as synarthroses joints, amphiarthroses joints, diarthroses joints, and the like thereof. As a further non-limiting example, arthritic group 116 may include one or more sutures, syndesmoses, and/or gomphoses groups. In an embodiment, and without limitation, arthritic group 116 may be identified as a function of user-entered data; as a non-limiting example, a user may answer a questionnaire by indicating there is pain in the user's hip. As a further non-limiting example, arthritic group 116 may be identified as a function of a user medical history that identifies a previous injury to a joint and/or connective tissue.

Still referring to FIG. 1, arthritic group 116 is identified as a function of a medical database 120. As used in this disclosure a “medical database” is a database containing one or more arthritic groups. Medical database 120 may be implemented, without limitation, as a relational databank, a key-value retrieval databank such as a NOSQL databank, or any other format or structure for use as a databank that a person skilled in the art would recognize as suitable upon review of the entirety of this disclosure. Medical database 120 may alternatively or additionally be implemented using a distributed data storage protocol and/or data structure, such as a distributed hash table or the like. Medical database 120 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Medical database 120 may include a peer review. Peer review may identify one or more arthritic groups as a function of a peer review evaluation conducted by one or more informed advisors with similar competencies. As a non-limiting example peer review may include professional peer reviews, scholarly peer reviews, government peer reviews, medical peer reviews, technical peer reviews, and the like thereof. Medical database 120 may include an informed advisor association. Informed advisor association may identify one or more arthritic groups as a function of one or more committees, organizations, and/or groups that at least determine and/or organize arthritic groups. As a non-limiting example informed advisor association may include the American Medical Association, the American College of Rheumatology, the National Psoriasis Foundation, the Arthritis Foundation, the Road Back Foundation, the Lupus Foundation of America, the Fibromyalgia Network, the American Lyme Disease Foundation, the Scoliosis Research Society, and the like thereof. Medical database 120 may include a medical website. Medical website may identify one or more arthritic groups as a function of one or more online and/or web-based medical recommendations. As a non-limiting example medical website may include Medline Plus, Drugs.com, Mayo Clinic, Orphanet, Medgadget, WebMD, Health.gov, SPM ePatients blog, and the like thereof.

Still referring to FIG. 1, computing device 104 produces arthritic batch 112 as a function of arthritic group 116 and arthritic element 108 using an arthritic machine-learning model 124. As used in this disclosure an “arthritic machine-learning model” is a machine-learning model to produce an arthritic batch output given arthritic groups and/or arthritic elements as inputs; this is in contrast to a non-machine learning software program where the commands to be executed are determined in advance by a user and written in a programming language. Arthritic machine-learning model 124 may include one or more arthritic machine-learning processes such as supervised, unsupervised, or reinforcement machine-learning processes that computing device 104 and/or a remote device may or may not use in the determination of arthritic batch 112. As used in this disclosure “remote device” is an external device to computing device 104. Arthritic machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train arthritic machine-learning process as a function of an arthritic training set. As used in this disclosure “arthritic training set” is a training set that correlates an arthritic group and/or arthritic element to an arthritic batch. For example, and without limitation, an arthritic group of a ball and socket joint in the shoulder and an arthritic element of swelling and/or pain of the joint may relate to an arthritic batch of a reduced function of the shoulder. The arthritic training set may be received as a function of user-entered valuations of arthritic groups, arthritic elements, and/or arthritic batches. Computing device 104 may receive arthritic training set by receiving correlations of arthritic groups, and/or arthritic elements that were previously received and/or determined during a previous iteration of determining arthritic batches. The arthritic training set may be received by one or more remote devices that at least correlate an arthritic group and/or arthritic element to an arthritic batch. The arthritic training set may be received in the form of one or more user-entered correlations of an arthritic group and/or arthritic element to an arthritic batch. Additionally or alternatively, a user may include an informed advisor, wherein an informed advisor may include, without limitation, family physicians, primary care physicians, orthopedists, rheumatologists, neurologists, dermatologists, occupational therapists, psychologists, psychiatrists, infectious disease physicians, geneticists, physical therapists, and the like thereof.

Still referring to FIG. 1, computing device 104 may receive arthritic machine-learning model 124 from a remote device that utilizes one or more arthritic machine learning processes, wherein a remote device is described above in detail. For example, and without limitation, a remote device may include a computing device, external device, processor, and the like thereof. Remote device may perform the arthritic machine-learning process using the arthritic training set to generate arthritic batch 112 and transmit the output to computing device 104. Remote device may transmit a signal, bit, datum, or parameter to computing device 104 that at least relates to arthritic batch 112. Additionally or alternatively, the remote device may provide an updated machine-learning model. For example, and without limitation, an updated machine-learning model may be comprised of a firmware update, a software update, an arthritic machine-learning process correction, and the like thereof. As a non-limiting example a software update may incorporate a new arthritic group that relates to a modified arthritic element. Additionally or alternatively, the updated machine learning model may be transmitted to the remote device, wherein the remote device may replace the arthritic machine-learning model with the updated machine-learning model and determine the arthritic batch as a function of the arthritic group using the updated machine-learning model. The updated machine-learning model may be transmitted by the remote device and received by computing device 104 as a software update, firmware update, or corrected arthritic machine-learning model. For example, and without limitation arthritic machine-learning model 112 may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

Still referring to FIG. 1, computing device 104 may produce arthritic batch 112 as a function of a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

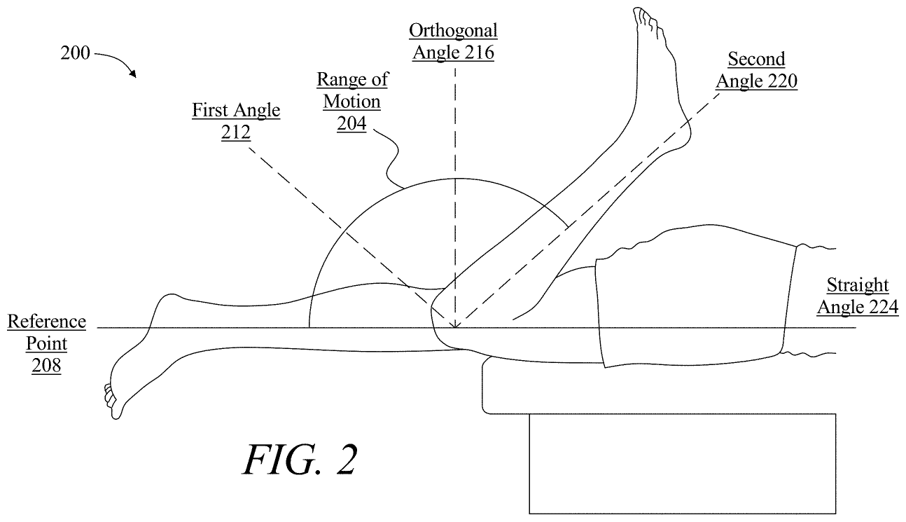

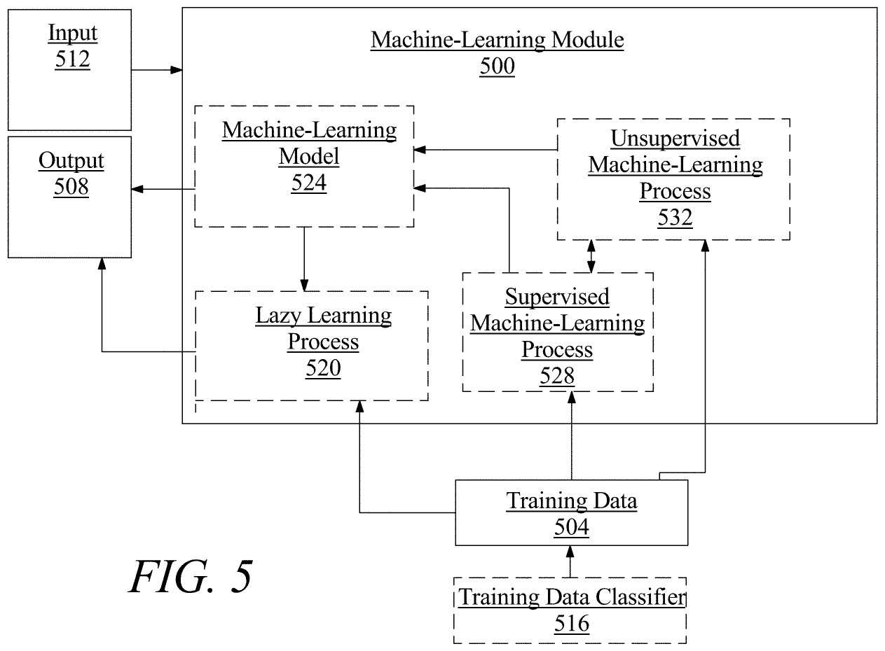

Still referring to FIG. 1, computing device 104 may produce arthritic batch 112 by identifying an arthritic enumeration. As used in this disclosure an “arthritic enumeration” is a measurable value associated with an effect of arthritis on an individual. For example, arthritic enumeration may be 12 for an individual with few symptoms associated with osteoarthritis. As a further non-limiting example, arthritic enumeration may be 74 for an individual experiencing joint locking associated with rheumatoid arthritis. Computing device 104 may identify arthritic enumeration as a function of receiving a user range of motion. As used in this disclosure a “user range of motion” is a range of motion a user is capable of performing for at least a joint, wherein a range of motion is described in detail below, in reference to FIG. 2. For example, and without limitation, a user range of motion may include a user abducting a shoulder 32°. As a further, non-limiting example, a user range of motion may include a user extending a knee 74°. Computing device 104 may determine a joint range of motion. As used in this disclosure a “joint range of motion” is a maximum range of motion a joint is capable of performing. For example, and without limitation, joint range of motion may include a range of 0° to 180° for a flexion movement of the shoulder. As a further non-limiting example joint range of motion may include 0° to 25° for an abduction movement for a wrist. Computing device 104 may determine arthritic enumeration as a function of user range of motion, joint range of motion, and an enumeration threshold. As used in this disclosure an “enumeration threshold” is a measurable value that represents a limit that the user range of motion may or may not exceed. In an embodiment, enumeration threshold may be obtained as a function of a medical database, wherein a medical database is described above in detail and/or a user entered value. In yet another embodiment, enumeration threshold may be obtained as a function of a query denoting a plurality of factors of an individual. For example and without limitation, one or more factors of an individual may include age, sex, height, weight, income, physical activity, demographics, and the like thereof. As a further non-limiting example, one or more factors may include one or more body types, such as but not limited to an ectomorph type, mesomorph type, endomorph type, and the like thereof. In an embodiment, enumeration threshold may be obtained as a function of one or more physical histories. For example, and without limitation, enumeration threshold may be obtained as a function of a previously playing a sport such as, but not limited to soccer, football, baseball, basketball, wrestling, lacrosse, volleyball, softball, fencing, running, and the like thereof. As a further non-limiting example, enumeration threshold may be obtained as a function of performing a hobby that requires physical activity, such as kayaking, rowing, windsurfing, hiking, and the like thereof. As a non-limiting example, computing device 104 may determine arthritic enumeration as a function of entering a user range of motion, joint range of motion, and enumeration threshold as inputs into an enumeration machine-learning model, wherein the enumeration machine-learning model outputs arthritic enumeration. Additionally or alternatively, enumeration machine-learning model may receive one or more inputs as a function of the query denoting the plurality of factors of an individual. For example, and without limitation enumeration machine-learning model may receive one or more inputs associated with a user range of motion for a 56-year-old woman that hikes, wherein the joint range of motion relates to a knee. Enumeration machine-learning model includes any of the machine-learning models as described below in detail, in reference to FIG. 5. As a further non-limiting example, computing device 104 may determine arthritic enumeration as a function of an enumeration formula, wherein an enumeration formula calculates arthritic enumeration as a function of a user range of motion, joint range of motion, and enumeration threshold. Enumeration machine-learning model includes any of the machine-learning models as described below in detail, in reference to FIG. 5. In an embodiment, and without limitation, enumeration formula may be produced as a function of enumeration machine-learning model. For example, and without limitation, arthritic enumeration may be 72 for a user that has a 10° range of motion in a shoulder, wherein enumeration threshold may indicate that a user is experiencing joint locking if the user range of motion is less than 20°, wherein the shoulder joint range of motion may have a possibility of 0° to 180°. As a further non-limiting example, arthritic enumeration may be 43 for a user that has a 72° range of motion in a hip, wherein enumeration threshold may indicate that a user is experiencing pain and/or swelling if the user range is greater than 40° but less than 90°, wherein the hip joint range of motion may have a possibility of 0° to 120°.

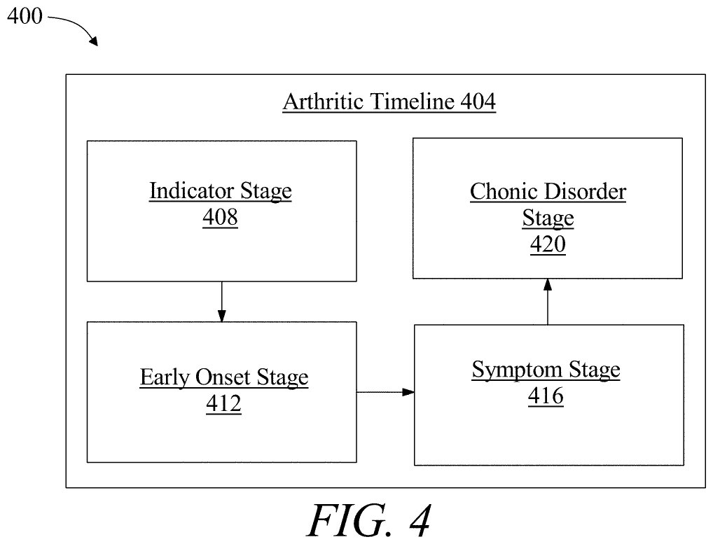

Still referring to FIG. 1, computing device 104 may produce arthritic batch 112 by receiving an arthritic timeline, wherein an arthritic timeline is a list and/or linear representation of events associated with arthritis during a time period, described in detail below in reference to FIG. 4. For example, and without limitation arthritic timeline may include one or more indicator stages, early-onset stages, symptom stages, and/or chronic disorder stages. Arthritic timeline may be received as a function of an arthritic database. As used in this disclosure an “arthritic database” is a database containing one or more data entries associated with the timeline of one or more types of arthritis. For example, and without limitation, arthritic database may include one or more medical websites, medical journals, medical textbooks, medical blog posts, medical records, and the like thereof. Computing device 104 may determine a progression parameter as a function of arthritic timeline and/or arthritic element. As used in this disclosure a “progression parameter” is a parameter that denotes a location on the timeline at which the user may be placed. For example, and without limitation, progression parameter may denote that a user is in the symptom stage of the arthritic timeline as a function of joint swelling and/or joint pain. As a further non-limiting example, progression parameter may indicate that a user has a high likelihood for the next progressive step in the arthritic timeline to include bone spurs, reduced muscle tension, reduced range of motion, and the like thereof.

In an embodiment and still referring to FIG. 1, arthritic batch 112 may be produced as a function of identifying a development vector. As used in this disclosure a “development vector” is a data structure that represents one or more a quantitative values and/or measures probability associated with developing arthritis. For example, and without limitation, development vector may indicate that an individual's hallux has a high probability of developing gout. As a further non-limiting example, development vector may indicate that an individual's ankle has a high likelihood of developing cartilage deterioration. In an embodiment, development vector may include one or more values associated with an individual's habits. For example, and without limitation, development vector may be 71 for an individual that cracks their knuckles. As a further non-limiting example, development vector may be 32 for an individual that wears high heels. As a further non-limiting example, development vector may be 27 for an individual that sleeps on their stomach. As a further non-limiting example, development vector may be 88 for an individual that is obese and/or overweight.

In an embodiment, and still referring to FIG. 1, a vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes.

Still referring to FIG. 1, computing device 104 may produce arthritic batch 112 by identifying an arthritic disorder. As used in this disclosure “arthritic disorder” is an ailment and/or collection of ailments that impact an individual's joints and/or connective tissues. As a non-limiting example, arthritic disorder may include osteoarthritis, rheumatoid arthritis, childhood arthritis, fibromyalgia, gout, Lupus, psoriasis, infectious arthritis, and the like thereof. Arthritic disorder may be identified as a function of one or more disorder machine-learning models. As used in this disclosure “disorder machine-learning model” is a machine-learning model to produce an arthritic disorder output given arthritic elements as inputs; this is in contrast to a non-machine learning software program where the commands to be executed are determined in advance by a user and written in a programming language. Disorder machine-learning model may include one or more disorder machine-learning processes such as supervised, unsupervised, or reinforcement machine-learning processes that computing device 104 and/or a remote device may or may not use in the determination of arthritic disorder. A disorder machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train disorder machine-learning process as a function of a disorder training set. As used in this disclosure “disorder training set” is a training set that correlates at least a health system effect and arthritic element 108 to an arthritic disorder. As used in this disclosure “health system effect” is an impact and/or effect on the joints and/or connective tissues of an individual. As a non-limiting example an arthritic element of swollen joints and a health system effect of reduced range of motion may relate to an arthritic disorder of rheumatoid arthritis. The disorder training set may be received as a function of user-entered valuations of arthritic elements, health system effects, and/or arthritic disorders. Computing device 104 may receive disorder training by receiving correlations of arthritic elements and/or health system effects that were previously received and/or determined during a previous iteration of determining arthritic disorders. The disorder training set may be received by one or more remote devices that at least correlate arthritic elements and/or health system effects to arthritic disorders, wherein a remote device is an external device to computing device 104, as described above. The disorder training set may be received by one or more user-entered correlations of arthritic elements and health system effects to arthritic disorders. Additionally or alternatively, a user may include an informed advisor, wherein an informed advisor may include, without limitation, family physicians, primary care physicians, orthopedists, rheumatologists, neurologists, dermatologists, occupational therapists, psychologists, psychiatrists, infectious disease physicians, geneticists, physical therapists, and the like thereof.

Still referring to FIG. 1, computing device 104 may receive disorder machine-learning model from a remote device that utilizes one or more disorder machine learning processes, wherein a remote device is described above in detail. For example, and without limitation, a remote device may include a computing device, external device, processor, and the like thereof. Remote device may perform the disorder machine-learning process using the disorder training set to generate arthritic disorder and transmit the output to computing device 104. Remote device may transmit a signal, bit, datum, or parameter to computing device 104 that at least relates to arthritic disorders. Additionally or alternatively, the remote device may provide an updated machine-learning model. For example, and without limitation, an updated machine-learning model may be comprised of a firmware update, a software update, a disorder machine-learning process correction, and the like thereof. As a non-limiting example a software update may incorporate a new arthritic element that relates to a modified health system effect. Additionally or alternatively, the updated machine learning model may be transmitted to the remote device, wherein the remote device may replace the disorder machine-learning model with the updated machine-learning model and determine the arthritic disorder as a function of the arthritic element using the updated machine-learning model. The updated machine-learning model may be transmitted by the remote device and received by computing device 104 as a software update, firmware update, or corrected disorder machine-learning model. For example, and without limitation arthritic machine-learning model may utilize a neural net machine-learning process, wherein the updated machine-learning model may incorporate hierarchical clustering machine-learning process.

In an embodiment and still referring to FIG. 1, computing device 104 may produce arthritic batch 112 as a function of determining an autoimmune disorder. As used in this disclosure an “autoimmune disorder” is an ailment that affects and/or has a likelihood of causing a human body's immune system to attack and/or destroy healthy body tissues by mistake. For example, an autoimmune disorder may include, but is not limited to, rheumatoid arthritis, diabetes, celiac disorder, inflammatory bowel syndrome, systemic lupus erythematosus, Sjogren's syndrome, multiple sclerosis, polymyalgia rheumatica, ankylosing spondylitis, alopecia areata, vasculitis, temporal arteritis, psoriasis, Guillain-Barre syndrome, aplastic anemia, chronic inflammatory demyelinating polyneuropathy, rheumatoid arthritis, and the like. Autoimmune disorder may include any autoimmune disorder used as an autoimmune disorder as described in U.S. Nonprovisional application Ser. No. 17/007,318, filed on Aug. 31, 2020, and entitled “SYSTEM AND METHOD FOR REPRESENTING AN ARRANGED LIST OF PROVIDER ALIMENT POSSIBILITIES,” the entirety of which is incorporated herein by reference.

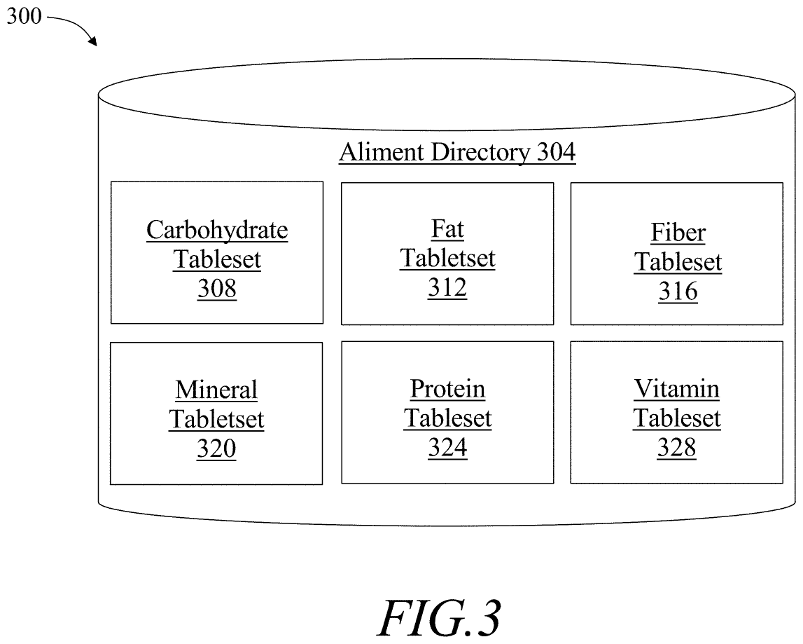

Still referring to FIG. 1, computing device 104 determines an edible 128 as a function of arthritic batch 112. Still referring to FIG. 1, computing device determines an edible 128 as a function of arthritic batch 112. As used in this disclosure an “edible” is a source of nourishment that may be consumed by a user such that the user may absorb the nutrients from the source. For example and without limitation, an edible may include legumes, plants, fungi, nuts, seeds, breads, dairy, eggs, meat, cereals, rice, seafood, desserts, dried foods, dumplings, pies, noodles, salads, stews, soups, sauces, sandwiches, and the like thereof. In an embodiment, edible may be determined as a function of progression parameter, wherein a first edible may be identified for a first progression parameter and a second edible may be determined for a second parameter. For example, and without limitation, a first edible of broccoli may be determined for a first progression parameter of swelling, wherein a second edible of salmon may be identified for a second progression parameter of joint locking and/or bone spurs. Computing device 104 may determine edible 128 as a function of receiving a nourishment composition. As used in this disclosure a “nourishment composition” is a list and/or compilation of all of the nutrients contained in an edible. As a non-limiting example nourishment composition may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof. Nourishment composition may be obtained as a function of an edible directory, wherein an “edible directory” is a database of edibles that may be identified as a function of one or more metabolic components, as described in detail below, in reference to FIG. 3.

Still referring to FIG. 1, computing device 104 may produce a nourishment demand as a function of arthritic batch 112. As used in this disclosure a “nourishment demand” is requirement and/or necessary amount of nutrients required for a user to consume. As a non-limiting example, nourishment demand may include a user requirement of 1,000 IU of vitamin D to be consumed per day. Nourishment demand may be determined as a function of receiving a nourishment goal. As used in this disclosure a “nourishment goal” is a recommended amount of nutrients that a user should consume. Nourishment goal may be identified by one or more organizations that relate to, represent, and/or study arthritis in humans, such as the American Medical Association, the American College of Rheumatology, the National Psoriasis Foundation, the Arthritis Foundation, the Road Back Foundation, the Lupus Foundation of America, the Fibromyalgia Network, the American Lyme Disease Foundation, the Scoliosis Research Society, and the like thereof.

Still referring to FIG. 1, computing device 104 identifies edible 128 as a function of nourishment composition, nourishment demand, and an edible machine-learning model. As used in this disclosure a “edible machine-learning model” is a machine-learning model to produce an edible output given nourishment compositions and nourishment demands as inputs; this is in contrast to a non-machine learning software program where the commands to be executed are determined in advance by a user and written in a programming language. Edible machine-learning model may include one or more edible machine-learning processes such as supervised, unsupervised, or reinforcement machine-learning processes that computing device 104 and/or a remote device may or may not use in the determination of edible 128, wherein a remote device is an external device to computing device 104 as described above in detail. An edible machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train edible machine-learning process as a function of an edible training set. As used in this disclosure an “edible training set” is a training set that correlates at least nourishment composition and nourishment demand to an edible. For example, and without limitation, nourishment composition of 32 g of fiber and a nourishment demand of 25 g of fiber as a function of a childhood arthritis may relate to an edible of amaranth. The edible training set may be received as a function of user-entered valuations of nourishment compositions, nourishment demands, and/or edibles. Computing device 104 may receive edible training set by receiving correlations of nourishment compositions and/or nourishment demands that were previously received and/or determined during a previous iteration of determining edibles. The edible training set may be received by one or more remote devices that at least correlate a nourishment composition and nourishment demand to an edible, wherein a remote device is an external device to computing device 104, as described above. Edible training set may be received in the form of one or more user-entered correlations of a nourishment composition and/or nourishment demand to an edible. Additionally or alternatively, a user may include an informed advisor, wherein an informed advisor may include, without limitation family physicians, primary care physicians, orthopedists, rheumatologists, neurologists, dermatologists, occupational therapists, psychologists, psychiatrists, infectious disease physicians, geneticists, physical therapists, and the like thereof.

Still referring to FIG. 1, computing device 104 may receive edible machine-learning model from a remote device that utilizes one or more edible machine learning processes, wherein remote device is described above in detail. For example, and without limitation, remote device may include a computing device, external device, processor, and the like thereof. Remote device may perform the edible machine-learning process using the edible training set to generate edible 128 and transmit the output to computing device 104. Remote device may transmit a signal, bit, datum, or parameter to computing device 104 that at least relates to edible 128. Additionally or alternatively, the remote device may provide an updated machine-learning model. For example, and without limitation, an updated machine-learning model may be comprised of a firmware update, a software update, an edible machine-learning process correction, and the like thereof. As a non-limiting example a software update may incorporate a new nourishment composition that relates to a modified nourishment demand. Additionally or alternatively, the updated machine learning model may be transmitted to the remote device, wherein the remote device may replace the edible machine-learning model with the updated machine-learning model and determine the edible as a function of the nourishment demand using the updated machine-learning model. The updated machine-learning model may be transmitted by the remote device and received by computing device 104 as a software update, firmware update, or corrected edible machine-learning model. For example, and without limitation an edible machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, edible machine-learning model may identify edible 128 as a function of one or more classifiers, wherein a classifier is described above in detail.

Still referring to FIG. 1, computing device 104 may identify edible as a function of a likelihood parameter. As used in this disclosure a “likelihood parameter” is a parameter that identities the probability of a user to consume an edible. As a non-limiting example likelihood parameter may identify a high probability that a user will consume an edible of broccoli. As a further non-limiting example likelihood parameter may identify a low probability that a user will consume an edible of Brussels sprouts. Likelihood parameter may be determined as a function of a user taste profile. As used in this disclosure a “user taste profile” is a profile of a user that identifies one or more desires, preferences, wishes, and/or wants that a user has. As a non-limiting example a user taste profile may include a user's preference for vanilla flavor and/or soft textured edibles. Likelihood parameter may be determined as a function of an edible profile. As used in this disclosure an “edible profile” is taste of an edible is the sensation of flavor perceived in the mouth and throat on contact with the edible. Edible profile may include one or more flavor variables. As used in this disclosure a “flavor variable” is a variable associated with the distinctive taste of an edible, wherein a distinctive may include, without limitation sweet, bitter, sour, salty, umami, cool, and/or hot. Edible profile may be determined as a function of receiving flavor variable from a flavor directory. As used in this disclosure a “flavor directory” is a database or other data structure including flavors for an edible. As a non-limiting example flavor directory may include a list and/or collection of edibles that all contain salty flavor variables. As a further non-limiting example flavor directory may include a list and/or collection of edibles that all contain sour flavor variables. Flavor directory may be implemented similarly to an edible directory as described below in detail, in reference to FIG. 3. Likelihood parameter may alternatively or additionally include any user taste profile and/or edible profile used as a likelihood parameter as described in U.S. Nonprovisional application Ser. No. 17/032,080, filed on Sep. 25, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

Still referring to FIG. 1, computing device 104 generates a nourishment program 132 as a function of edible 128. As used in this disclosure a “nourishment program” is a program consisting of one or more edibles that are to be consumed over a given time period, wherein a time period is a temporal measurement such as seconds, minutes, hours, days, weeks, months, years, and the like thereof. As a non-limiting example nourishment program 132 may consist of recommending broccoli for 8 days. As a further non-limiting example nourishment program 132 may recommend fish for a first day, citrus fruits for a second day, and garlic for a third day. Nourishment program 132 may include one or more diet programs such as paleo, keto, vegan, vegetarian, Mediterranean, Dukan, Zone, HCG, and the like thereof. Computing device 104 may develop nourishment program 132 as a function of an arthritic outcome. As used in this disclosure an “arthritic outcome” is an outcome that an edible may generate according to a predicted and/or purposeful plan. As a non-limiting example, arthritic outcome may include a treatment outcome. As used in this disclosure a “treatment outcome” is an intended outcome that is designed to at least reverse and/or eliminate arthritic batch 112, arthritic element 108, and/or arthritic disorder. As a non-limiting example, a treatment outcome may include reversing the effects of the arthritic disorder fibromyalgia. As a further non-limiting example, a treatment outcome includes reversing the arthritic disorder of gout. Arthritic outcome may include a prevention outcome. As used in this disclosure a “prevention outcome” is an intended outcome that is designed to at least prevent and/or avert arthritic batch 112, arthritic element 108, and/or arthritic disorder. As a non-limiting example, a prevention outcome may include preventing the development of the arthritic disorder rheumatoid arthritis.

Still referring to FIG. 1, computing device 104 may develop nourishment program 132 as a function of edible 128 and arthritic outcome using a nourishment machine-learning model. As used in this disclosure a “nourishment machine-learning model” is a machine-learning model to produce a nourishment program output given edibles and/or arthritic outcomes as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the development of nourishment program 132. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates an arthritic outcome to an edible. The nourishment training set may be received as a function of user-entered edibles, arthritic outcomes, and/or nourishment programs. For example, and without limitation, an arthritic outcome of treating osteoarthritis may correlate to an edible of yogurt. Computing device 104 may receive nourishment training by receiving correlations of arthritic outcomes and/or edibles that were previously received and/or determined during a previous iteration of developing nourishment programs. The nourishment training set may be received by one or more remote devices that at least correlate an arthritic outcome and/or edible to a nourishment program, wherein a remote device is an external device to computing device 104, as described above. Nourishment training set may be received in the form of one or more user-entered correlations of an arthritic outcome and/or edible to a nourishment program. Additionally or alternatively, a user may include an informed advisor, wherein an informed advisor may include, without limitation family physicians, primary care physicians, orthopedists, rheumatologists, neurologists, dermatologists, occupational therapists, psychologists, psychiatrists, infectious disease physicians, geneticists, physical therapists, and the like thereof.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to develop nourishment program 132 and transmit the output to computing device 104. The remote device may transmit a signal, bit, datum, or parameter to computing device 104 that at least relates to nourishment program 132. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new arthritic outcome that relates to a modified edible. Additionally or alternatively, the updated machine learning model may be transmitted to the remote device, wherein the remote device may replace the nourishment machine-learning model with the updated machine-learning model and develop the nourishment program as a function of the arthritic outcome using the updated machine-learning model. The updated machine-learning model may be transmitted by the remote device and received by computing device 104 as a software update, firmware update, or corrected nourishment machine-learning model. For example, and without limitation nourishment machine-learning model may utilize a neural net machine-learning process, wherein the updated machine-learning model may incorporate decision tree machine-learning processes.

Now referring to FIG. 2, an exemplary embodiment 200 of a range of motion 204 is illustrated. As used in this disclosure a “range of motion” is a full movement potential of a joint. For example, and without limitation, range of motion 204 may include one or more ranges of joint movement such as, but not limited to, flexion, extension, abduction, adduction, medial rotation, lateral rotation, elevation, depression, pronation, supination, dorsiflexion, plantar flexion, inversion, eversion, opposition, reposition, circumduction, protraction, retraction, and the like thereof. For example, and without limitation range of motion 204 may include a range of 0° to 125° for flexion of a hip joint. As a further non-limiting example, range of motion 204 may include a range of 120° to 0° for extension of a knee joint. As a further non-limiting example, range of motion 204 may include a range of 0° to 20° for dorsiflexion of an ankle joint. As a further non-limiting example, range of motion 204 may include a range of 0° to 80° for extension of a metatarsophalangeal joint. As a further non-limiting example, range of motion 204 may include a range of 0° to 50° for flexion of an interphalangeal joint of the toe. As a further non-limiting example, range of motion 204 may include a range of 0° to 90° for abduction of a shoulder joint. As a further non-limiting example, range of motion 204 may include a range of 0° to 90° for supination of an elbow joint. As a further non-limiting example, range of motion 204 may include a range of 0° to 65° for adduction of a wrist joint.

Still referring to FIG. 2, range of motion 204 may include a reference point 208. As used in this disclosure a “reference point” is a point and/or direction of reference for the range of motion to originate from. For example a reference point may include a direction in a planar coordinate system, such as 0°, 52°, 113°, 270°, 300°, and the like thereof. As a further non-limiting example, referent point may include a point and/or direction in a spherical coordinate system. Range of motion 204 may include a first angle 212. As used in this disclosure a “first angle” is a first angular movement from the reference point. For example, and without limitation, first angular movement may include 0°, 22°, 37°, 45°, 82°, and the like thereof. First angle 212 may include any angular movement between reference point and an orthogonal angle 216. As used in this disclosure an “orthogonal angle” is an angle that is perpendicular to the reference point. For example and without limitation, orthogonal angle 216 may include an angle of 90° and/or 270°. Range of motion 204 may include a second angle 220. As used in this disclosure a “second angle” is a second angular movement from the reference point. For example, and without limitation, second angular movement may include 97°, 119°, 142°, 163°, 178°, and the like thereof. Second angle 220 may include any angular movement between reference point and a straight angle 224. As used in this disclosure a “straight angle” is an angle whose sides lie in opposite directions from the orthogonal angle in the same straight line. For example and without limitation straight angle 224 may include an angle of 180°.

Now referring to FIG. 3, an exemplary embodiment 300 of an edible directory 304 according to an embodiment of the invention is illustrated. Edible directory 304 may be implemented, without limitation, as a relational databank, a key-value retrieval databank such as a NOSQL databank, or any other format or structure for use as a databank that a person skilled in the art would recognize as suitable upon review of the entirety of this disclosure. Edible directory 304 may alternatively or additionally be implemented using a distributed data storage protocol and/or data structure, such as a distributed hash table or the like. Edible directory 304 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Edible directory 304 may include a carbohydrate tableset 308. Carbohydrate tableset 308 may relate to a nourishment composition of an edible with respect to the quantity and/or type of carbohydrates in the edible. As a non-limiting example, carbohydrate tableset 308 may include monosaccharides, disaccharides, oligosaccharides, polysaccharides, and the like thereof. Edible directory 304 may include a fat tableset 312. Fat tableset 312 may relate to a nourishment composition of an edible with respect to the quantity and/or type of esterified fatty acids in the edible. Fat tableset 312 may include, without limitation, triglycerides, monoglycerides, diglycerides, phospholipids, sterols, waxes, and free fatty acids. Edible directory 304 may include a fiber tableset 316. Fiber tableset 316 may relate to a nourishment composition of an edible with respect to the quantity and/or type of fiber in the edible. As a non-limiting example, fiber tableset 316 may include soluble fiber, such as beta-glucans, raw guar gum, psyllium, inulin, and the like thereof as well as insoluble fiber, such as wheat bran, cellulose, lignin, and the like thereof. Edible directory 304 may include a mineral tableset 320. Mineral tableset 320 may relate to a nourishment composition of an edible with respect to the quantity and/or type of minerals in the edible. As a non-limiting example, mineral tableset 320 may include calcium, phosphorous, magnesium, sodium, potassium, chloride, sulfur, iron, manganese, copper, iodine, zing, cobalt, fluoride, selenium, and the like thereof. Edible directory 304 may include a protein tableset 324. Protein tableset 324 may relate to a nourishment composition of an edible with respect to the quantity and/or type of proteins in the edible. As a non-limiting example, protein tableset 324 may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Edible directory 304 may include a vitamin tableset 328. Vitamin tableset 328 may relate to a nourishment composition of an edible with respect to the quantity and/or type of vitamins in the edible. As a non-limiting example, vitamin tableset 328 may include vitamin A, vitamin B₁, vitamin B₂, vitamin B₃, vitamin Bs, vitamin B₆, vitamin B₇, vitamin B₉, vitamin B₁₂, vitamin C, vitamin D, vitamin E, vitamin K, and the like thereof.

Now referring to FIG. 4, an exemplary embodiment 400 of an arthritic timeline 404 is illustrated. As used in this disclosure an “arthritic timeline” is a list and/or linear representation of events associated with arthritis during a time period, wherein a time period is a metric of time, such as, but not limited to, seconds, minutes, hours, days, weeks, months, years, decades and the like thereof. For example and without limitation, arthritic timeline may include a one or more event such as early-stage rheumatoid arthritis, moderate-stage rheumatoid arthritis, severe rheumatoid arthritis, and/or locked joint rheumatoid arthritis. Arthritic timeline 404 may include an indicator stage 408. As used in this disclosure an “indicator stage” is an event of that may indicate the likelihood of developing an arthritic disorder. For example, and without limitation, indicator stage 408 may include general weakness of a joint, dry mouth, weight loss, loss of appetite, hard bumps of tissue under the skin, chest pain, eye discharge, and the like thereof. Arthritic timeline 404 may include an early-onset stage 412. As used in this disclosure an “early-onset stage” is an event of that indicates the development of an arthritic disorder. For example, and without limitation early-onset stage 412 may include redness, fever, joint stiffness, lethargy, and the like thereof. Arthritic timeline 404 may include a symptom stage 416. As used in this disclosure a “symptom stage” is an event of an arthritic disorder wherein the user is displaying and/or experiencing symptoms. For example, and without limitation symptom stage 416 may include decreased range of motion, swelling of the joints, joint pain, and the like thereof. Arthritic timeline 404 may include a chronic stage 420. As used in this disclosure a chronic stage” is an event of recurring and/or repeated symptoms associated with an arthritic disorder that compounds. For example, and without limitation chronic stage 420 may include lack of motion in a joint and/or joint locking, bone erosion, joint deformity, and the like thereof.

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example inputs of arthritic elements and/or arthritic groups may result in an output of an arthritic batch.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to sub-categories of arthritic groups such as, but not limited to, a group of cells, tissues, and/or organs in a region of the body, such as but not limited to shoulders, knees, hands, wrists, and the like thereof.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include arthritic elements and/or arthritic groups as described above as inputs, arthritic batches as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

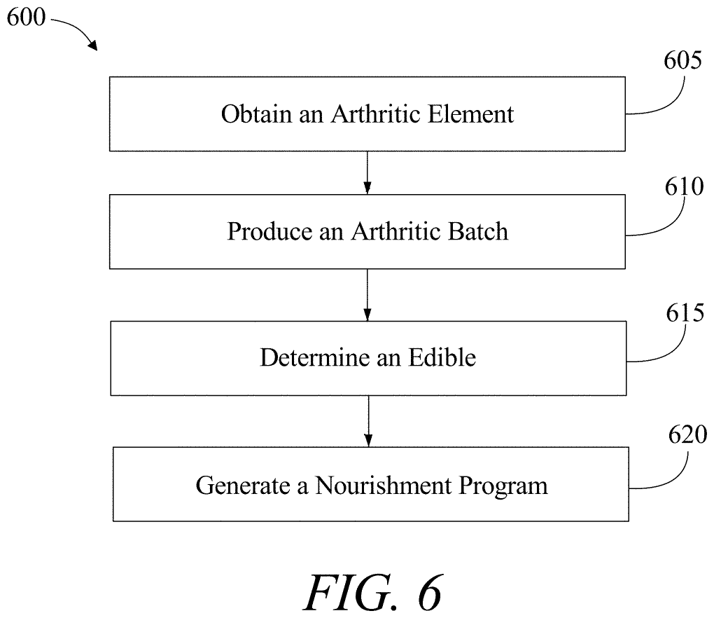

Now referring to FIG. 6, an exemplary embodiment of a method 600 for generating an arthritic disorder nourishment program is illustrated. At step 605, a computing device 104 obtains an arthritic element 108. Computing device 104 includes any of the computing device 104 as described above, in reference to FIGS. 1-5. Arthritic element 108 includes any of the arthritic element 108 as described above, in reference to FIGS. 1-5.

Still referring to FIG. 6, at step 610, computing device 104 produces an arthritic batch 112 as a function of arthritic element 108. Arthritic batch 112 includes any of the arthritic batch 112 as described above, in reference to FIGS. 1-5. Computing device 104 produces arthritic batch 112 by identifying an arthritic group 116 as a function of a medical database 120. Arthritic group 116 includes any of the arthritic group 116 as described above, in reference to FIGS. 1-5. Medical database 120 includes any of the medical database 120 as described above, in reference to FIGS. 1-5. Computing device 104 produces arthritic batch 112 as a function of arthritic group 116 and arthritic element 108 using an arthritic machine-learning model 124. Arthritic machine-learning model 124 includes any of the arthritic machine-learning model 124 as described above, in reference to FIGS. 1-5.

Still referring to FIG. 6, at step 615, computing device 104 determines an edible 128 as a function of arthritic batch 112. Edible 128 includes any of the edible 128 as described above, in reference to FIGS. 1-5.

Still referring to FIG. 6, at step 620, computing device 104 generates a nourishment program 132 as a function of edible 128. Nourishment program 132 includes any of the nourishment program 132 as described above, in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

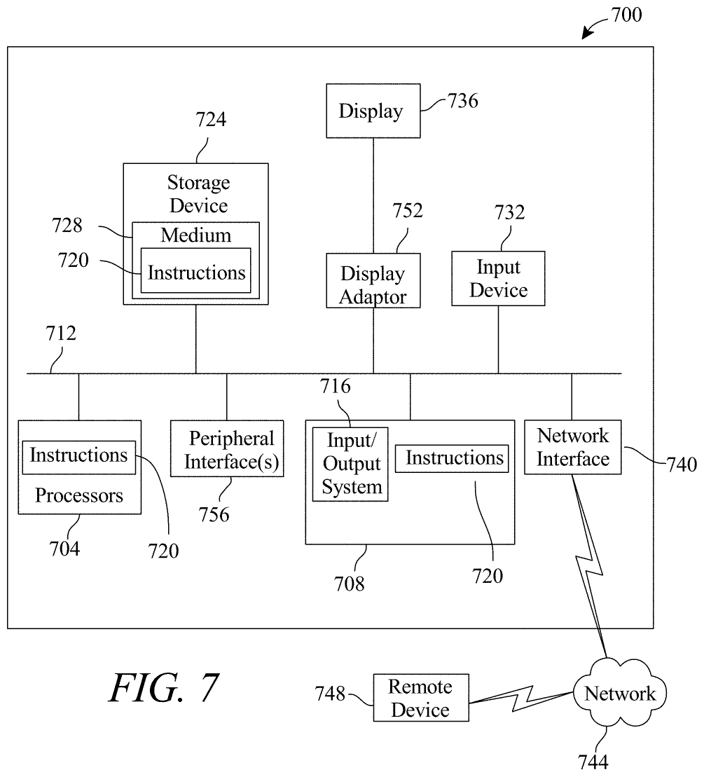

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating an arthritic disorder nourishment program, the system comprising:
a computing device, the computing device configured to:
obtain an arthritic element;
produce an arthritic batch as a function of the arthritic element, wherein producing the arthritic batch further comprises:
identifying an arthritic group as a function of a medical database; and
producing the arthritic batch as a function of the arthritic group and the arthritic element using an arthritic machine-learning model;
determine an edible as a function of the arthritic batch; and
generate a nourishment program as a function of the edible.

2. The system of claim 1, wherein the arthritic element includes a genetic element.

3. The system of claim 1, wherein obtaining the arthritic element includes receiving an arthritic questionnaire and obtaining the arthritic element as a function of the arthritic questionnaire.

4. The system of claim 1, wherein producing the arthritic batch further comprises identifying an arthritic enumeration and producing the arthritic batch as a function of the arthritic enumeration.

5. The system of claim 4, wherein identifying the arthritic enumeration further comprises:
receiving a user range of motion;
determining a joint range of motion; and
determining the arthritic enumeration as a function of the user range of motion, the joint range of motion, and an enumeration threshold.

6. The system of claim 1, wherein producing the arthritic batch includes determining an arthritic disorder and producing the arthritic batch as a function of the arthritic disorder.

7. The system of claim 1, wherein producing the arthritic batch includes determining an autoimmune disorder and producing the arthritic batch as a function of the autoimmune disorder.

8. The system of claim 1, wherein producing the arthritic batch further comprises:
identifying a development vector; and
producing the arthritic batch as a function of the development vector.

9. The system of claim 1, wherein producing the arthritic batch further comprises:
receiving an arthritic timeline;
determining a progression parameter as a function of the arthritic timeline and arthritic element; and
producing the arthritic batch as a function of the progression parameter.

10. The system of claim 1, wherein generating the nourishment program further comprises:
receiving an arthritic outcome; and
generating the nourishment program as a function of the arthritic outcome using a nourishment machine-learning model.

11. A method for generating an arthritic disorder nourishment program, the method comprising:
obtaining, by a computing device, an arthritic element;
producing, by the computing device, an arthritic batch as a function of the arthritic element, wherein producing the arthritic batch further comprises:
identifying an arthritic group as a function of a medical database; and
producing the arthritic batch as a function of the arthritic group and the arthritic element using an arthritic machine-learning model;
determining, by the computing device, an edible as a function of the arthritic batch; and
generating, by the computing device, a nourishment program as a function of the edible.

12. The method of claim 11, wherein the arthritic element includes a genetic element.

13. The method of claim 11, wherein obtaining the arthritic element includes receiving an arthritic questionnaire and obtaining the arthritic element as a function of the arthritic questionnaire.

14. The method of claim 11, wherein producing the arthritic batch further comprises identifying an arthritic enumeration and producing the arthritic batch as a function of the arthritic enumeration.

15. The method of claim 14, wherein identifying the arthritic enumeration further comprises:
receiving a user range of motion;
determining a joint range of motion; and
determining the arthritic enumeration as a function of the user range of motion, the joint range of motion, and an enumeration threshold.

16. The method of claim 11, wherein producing the arthritic batch includes determining an arthritic disorder and producing the arthritic batch as a function of the arthritic disorder.

17. The method of claim 11, wherein producing the arthritic batch includes determining an autoimmune disorder and producing the arthritic batch as a function of the autoimmune disorder.

18. The method of claim 11, wherein producing the arthritic batch further comprises:
identifying a development vector; and
producing the arthritic batch as a function of the development vector.

19. The method of claim 11, wherein producing the arthritic batch further comprises:
receiving an arthritic timeline;
determining a progression parameter as a function of the arthritic timeline and arthritic element; and
producing the arthritic batch as a function of the progression parameter.

20. The method of claim 11, wherein generating the nourishment program further comprises:
receiving an arthritic outcome; and
generating the nourishment program as a function of the arthritic outcome using a nourishment machine-learning model.
